(19) United States
(12) Patent Application Publication
(10) Pub. No.: US 2025/0137979 A1
(43) Pub. Date: May 1, 2025

(54) METHOD AND APPARATUS FOR PERFORMING SENSOR DRIFT CORRECTION BASED ON DOUBLE CYCLING MEASUREMENTS

(71) Applicant: Electronics and Telecommunications Research Institute, Daejeon (KR)

(72) Inventors: Hyung Wook NOH, Daejeon (KR); Do Hyeun KIM, Daejeon (KR); Hwin Dol PARK, Daejeon (KR); Chang Geun AHN, Daejeon (KR); Yong Won JANG, Daejeon (KR); Jae Hun CHOI, Daejeon (KR)

(21) Appl. No.: 18/927,774

(22) Filed: Oct. 25, 2024

(30) Foreign Application Priority Data
Oct. 25, 2023 (KR) 10-2023-0144131

(51) Int. Cl. G01N 33/00

(52) U.S. Cl. CPC G01N 33/0006

(57) ABSTRACT

The present disclosure relates to a method and apparatus for performing sensor drift compensation based on double cycling measurement. A method for performing drift correction according to gas sensor measurement according to an embodiment of the present disclosure may comprise: obtaining first measurement data for a reference gas for each sensor using one or more sensors in a first cycle; obtaining second measurement data for a target gas for each sensor using the one or more sensors in a second cycle; and generating a drift-corrected feature for each sensor based on a ratio calculated by dividing the second measurement data by the first measurement data.

### CROSS-REFERENCE TO RELATED APPLICATIONS

This application claims the benefit of earlier filing date and right of priority to Korean Application No. 10-2023-0144131, filed on Oct. 25, 2023, the contents of which are all hereby incorporated by reference herein in their entirety.

### TECHNICAL FIELD

The present disclosure relates to a method and device for performing sensor drift correction based on double cycling measurement and generating various characteristic features based thereon to improve the performance and reliability of a system.

### BACKGROUND

E-nose systems based on gas sensors have been continuously developed over the past several decades and are being applied in various industrial fields as a technology for detecting various volatile substances and recognizing patterns.

Existing gas sensor technologies have focused on the detection of specific gas components, and research has mainly been conducted with the goal of selective detection of one or a few gas components. However, in recent years, in various fields such as industrial fields, environment, and national defense safety, the target gases to be detected are becoming more diverse, and accordingly, the need for diverse sensor characteristics and feature diversification characteristics is increasing.

### SUMMARY

The technical object of the present disclosure is to provide a method and device for performing sensor drift correction based on double cycling measurement.

The technical object of the present disclosure relates to a method and device for performing sensor drift correction based on double cycling measurement and generating various characteristic features based thereon, thereby improving the performance and reliability of a system.

The technical object of the present disclosure relates to a method and device capable of solving a drift compensation problem of a sensor and accurately extracting characteristics for various gas components to improve the performance and reliability of a system.

The technical objects to be achieved by the present disclosure are not limited to the above-described technical objects, and other technical objects which are not described herein will be clearly understood by those skilled in the pertinent art from the following description.

A method for performing drift correction according to gas sensor measurement according to one aspect of the present disclosure may comprise: obtaining first measurement data for a reference gas for each sensor using one or more sensors in a first cycle; obtaining second measurement data for a target gas for each sensor using the one or more sensors in a second cycle; and generating a drift-corrected feature for each sensor based on a ratio calculated by dividing the second measurement data by the first measurement data.

An apparatus for performing drift correction according to gas sensor measurement according to an additional aspect of the present disclosure may comprise a processor and a memory, and the processor may be configured to obtain first measurement data for a reference gas for each sensor using one or more sensors in a first cycle; obtain second measurement data for a target gas for each sensor using the one or more sensors in a second cycle; and generate a drift-corrected feature for each sensor based on a ratio calculated by dividing the second measurement data by the first measurement data.

As one or more non-transitory computer readable medium storing one or more instructions according to an additional aspects, the one or more instructions may be executed by one or more processors and control an apparatus for performing drift correction based on gas sensor measurements to: obtain first measurement data for a reference gas for each sensor using one or more sensors in a first cycle; obtain second measurement data for a target gas for each sensor using the one or more sensors in a second cycle; and generate a drift-corrected feature for each sensor based on a ratio calculated by dividing the second measurement data by the first measurement data.

In various aspects of the present disclosure, a step/operation of generating one or more new features through combinations between drift-corrected features, in a case that a plurality of drift-corrected features for a plurality of sensors are generated may be added. In this regard, when n sensors exist and drift-corrected features for m sensors are selected for the combination, a maximum number of the one or more new features may correspond to ₙCₘ. Herein, ₓC_{y} represents a combination function for input x and input y.

Additionally, in various aspects of the present disclosure, a step/operation of generating one or more new features by applying an exponential function or a logarithmic function to the plurality of drift-corrected features in a case that a plurality of drift-corrected features are generated for a plurality of sensors may be added.

Additionally, in various aspects of the present disclosure, a step/operation of generating a polynomial for the plurality of drift-corrected features in a case that a plurality of drift-corrected features are generated for a plurality of sensors, and a step/operation of generating one or more new features based on an increased order according to the polynomial may be added.

Additionally, in various aspects of the present disclosure, a step/operation of generating one or more new features with reduced dimension by applying a pre-defined dimension reduction scheme to the plurality of drift-corrected features in a case that a plurality of drift-corrected features are generated for a plurality of sensors may be added.

Additionally, in various aspects of the present disclosure, a step/operation of clustering the plurality of drift-corrected features by applying a pre-defined clustering scheme in a case that a plurality of drift-corrected features are generated for a plurality of sensors, and a step/operation of generating one or more new features based on characteristics of data belonging to each cluster may be added.

Additionally, in various aspects of the present disclosure, a step/operation of generating one or more new features by applying a pre-defined neural network model or a pre-defined feature selection algorithm to the plurality of drift-corrected features in a case that a plurality of drift-corrected features are generated for a plurality of sensors may be added.

According to the present disclosure, a method and device for performing sensor drift correction based on double cycling measurement may be provided.

According to the present disclosure, a method and device may be provided that can improve the performance and reliability of a system by performing sensor drift correction based on double cycling measurement and generating various characteristic features based thereon.

According to the present disclosure, a method and device are provided that can solve a drift compensation problem of a sensor and accurately extract characteristics for various gas components to improve the performance and reliability of a system.

The effects obtainable from the present disclosure are not limited to the effects mentioned above, and other effects not mentioned will be clearly understood by a person skilled in the art to which the present disclosure belongs from the description below.

### BRIEF DESCRIPTION OF THE DRAWINGS

FIG. 1 illustrates various sensor-based measurement data for target targets to which the present disclosure may be applied.

FIG. 2 illustrates the drift effect of a gas sensor to which the present disclosure may be applied.

FIG. 3 illustrates a system configuration diagram for drift correction and feature combination according to an embodiment of the present disclosure.

FIG. 4 illustrates simulation results showing the drift correction effect according to the double cycling technique according to an embodiment of the present disclosure.

FIG. 5 illustrates the effect of drift correction based on double cycling obtained through data measured from a gas sensor according to an embodiment of the present disclosure.

FIG. 6 illustrates feature diversification and expansion effects according to combinations of sensor characteristic features according to an embodiment of the present disclosure.

FIG. 7 illustrates a simplified system configuration implementing a double cycling technique and feature combination according to an embodiment of the present disclosure.

FIG. 8 illustrates an operational flowchart for a drift correction and feature combination method according to an embodiment of the present disclosure.

FIG. 9 is a block diagram illustrating a device according to an embodiment of the present disclosure.

### DETAILED DESCRIPTION

As the present disclosure may make various changes and have multiple embodiments, specific embodiments are illustrated in a drawing and are described in detail in a detailed description. But, it is not to limit the present disclosure to a specific embodiment, and should be understood as including all changes, equivalents and substitutes included in an idea and a technical scope of the present disclosure. A similar reference numeral in a drawing refers to a like or similar function across multiple aspects. A shape and a size, etc. of elements in a drawing may be exaggerated for a clearer description. A detailed description on exemplary embodiments described below refers to an accompanying drawing which shows a specific embodiment as an example.

These embodiments are described in detail so that those skilled in the pertinent art can implement an embodiment. It should be understood that a variety of embodiments are different each other, but they do not need to be mutually exclusive. For example, a specific shape, structure and characteristic described herein may be implemented in other embodiment without departing from a scope and a spirit of the present disclosure in connection with an embodiment. In addition, it should be understood that a position or an arrangement of an individual element in each disclosed embodiment may be changed without departing from a scope and a spirit of an embodiment. Accordingly, a detailed description described below is not taken as a limited meaning and a scope of exemplary embodiments, if properly described, are limited only by an accompanying claim along with any scope equivalent to that claimed by those claims.

In the present disclosure, a term such as first, second, etc. may be used to describe a variety of elements, but the elements should not be limited by the terms. The terms are used only to distinguish one element from other element. For example, without getting out of a scope of a right of the present disclosure, a first element may be referred to as a second element and likewise, a second element may be also referred to as a first element. A term of and/or includes a combination of a plurality of relevant described items or any item of a plurality of relevant described items.

When an element in the present disclosure is referred to as being “connected” or “linked” to another element, it should be understood that it may be directly connected or linked to that another element, but there may be another element between them. Meanwhile, when an element is referred to as being “directly connected” or “directly linked” to another element, it should be understood that there is no another element between them.

As construction units shown in an embodiment of the present disclosure are independently shown to represent different characteristic functions, it does not mean that each construction unit is composed in a construction unit of separate hardware or one software. In other words, as each construction unit is included by being enumerated as each construction unit for convenience of a description, at least two construction units of each construction unit may be combined to form one construction unit or one construction unit may be divided into a plurality of construction units to perform a function, and an integrated embodiment and a separate embodiment of each construction unit are also included in a scope of a right of the present disclosure unless they are beyond the essence of the present disclosure.

A term used in the present disclosure is just used to describe a specific embodiment, and is not intended to limit the present disclosure. A singular expression, unless the context clearly indicates otherwise, includes a plural expression. In the present disclosure, it should be understood that a term such as “include” or “have”, etc. is just intended to designate the presence of a feature, a number, a step, an operation, an element, a part or a combination thereof described in the present specification, and it does not exclude in advance a possibility of presence or addition of one or more other features, numbers, steps, operations, elements, parts or their combinations. In other words, a description of “including” a specific configuration in the present disclosure does not exclude a configuration other than a corresponding configuration, and it means that an additional configuration may be included in a scope of a technical idea of the present disclosure or an embodiment of the present disclosure.

Some elements of the present disclosure are not a necessary element which performs an essential function in the present disclosure and may be an optional element for just improving performance. The present disclosure may be implemented by including only a construction unit which is necessary to implement essence of the present disclosure except for an element used just for performance improvement, and a structure including only a necessary element except for an optional element used just for performance improvement is also included in a scope of a right of the present disclosure.

Hereinafter, an embodiment of the present disclosure is described in detail by referring to a drawing. In describing an embodiment of the present specification, when it is determined that a detailed description on a relevant disclosed configuration or function may obscure a gist of the present specification, such a detailed description is omitted, and the same reference numeral is used for the same element in a drawing and an overlapping description on the same element is omitted.

Recently, considering various fields such as industrial fields, environment, and national defense safety, the target gases to be detected are becoming more diverse. Accordingly, the need for various sensor characteristics and the need for diversified feature characteristics are required.

Using various sensors may provide the following technical benefits in gas sensor-based systems.

First, it has the effect of detecting various gas components.

Specifically, by using various sensors, various types of gas components may be detected simultaneously. This enables accurate recognition of complex gas mixtures that may occur in real environments.

Additionally, there is an effect of improving selectivity.

Specifically, each sensor has high selectivity for a specific gas component. By combining various sensors, the selectivity for a specific gas component may be improved. Through this, the distinction and identification of gas components may be performed more accurately.

Additionally, there is an effect of improving reliability.

Specifically, by using various sensors, complementary characteristics between sensors may be utilized. By integrating independent measurement results of sensors, more reliable judgment and decision may be made. Through this, sensor errors and noises may be compensated, and the reliability of the system may be improved.

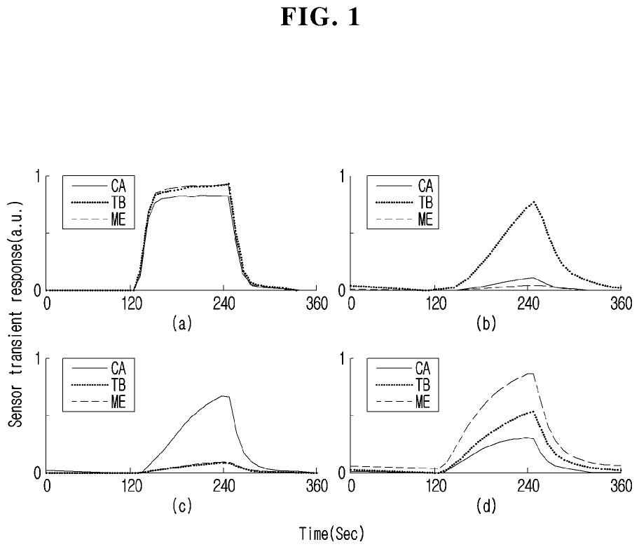

FIG. 1 illustrates various sensor-based measurement data for target targets to which the present disclosure may be applied.

Referring to FIG. 1, sensor-based measurements may be performed by setting target objects as cannabis (CA), methamphetamine (ME), and tobacco (TB). Additionally, (a), (b), (c), and (d) of FIG. 1 illustrate four different sensor-based measurement data.

Since the sensor (a) exhibits similar responsiveness, known as cross sensitivity ratio, to all three target targets, it may be difficult to distinguish between target gases.

Sensor (b) shows excellent selectivity for TB, and sensor (c) shows high reactivity for CA.

The sensor (d) exhibits high reactivity to ME and may also discriminate between the other two target gases.

Referring to the measurement data of sensors (a) to (d), the possibility of accurately detecting a specific drug in a tested sample may be increased by utilizing various sensors and features. This is because the approach may increase the probability of obtaining a feature that reacts very selectively to a specific drug.

The features described in the present disclosure may mean characteristics/properties of each sensor, such as responsiveness, discrimination, selectivity, etc.

Combining features through various combinations of various gas sensors may enhance the recognition ability for various gas mixtures. For example, in various applications such as industrial sites and environmental monitoring, there are frequent cases where simultaneous detection of various gas components is required.

Additionally, the diversified characteristics through combinations between features may further improve the selectivity and discrimination of the gas sensor, and thus, accurate and reliable results may be obtained.

Additionally, if the sensor has various features while each feature may be clearly distinguished and patterned, the reliability and usability of the sensor may be greatly improved.

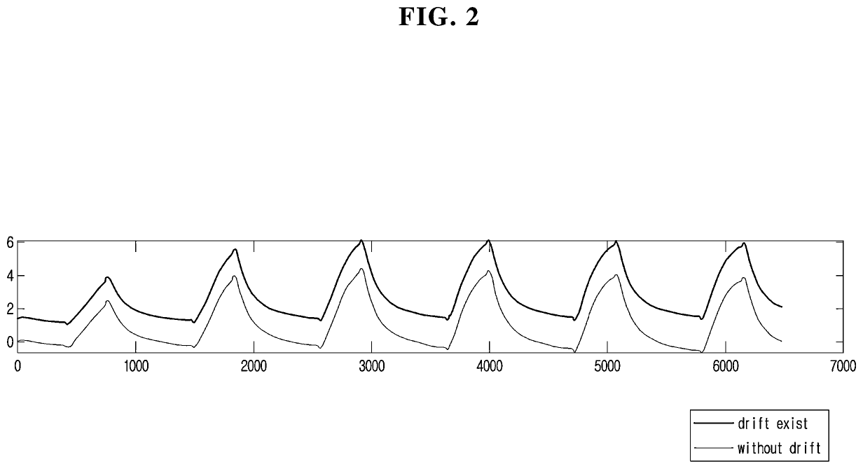

FIG. 2 illustrates the drift effect of a gas sensor to which the present disclosure may be applied.

Referring to FIG. 2, in the case of a gas sensor, a drift effect may occur in which the operating characteristics may change over time. Specifically, FIG. 2 shows the baseline characteristics of a signal when drift exists or does not exist.

For example, when referring to actual measured data based on a gas sensor, if a sensor drift effect occurs, a phenomenon such as a slight upward increase in the baseline characteristics of the signal may occur.

The aforementioned drift effect may affect the sensitivity, selectivity, response time, etc. of the sensor, which may result in a decrease in the reliability and accuracy of the sensor.

Therefore, compensation for drift effects may be essential to maintain long-term stability of gas sensors.

Various methods may be used for drift correction.

For example, in order to monitor the drift of the sensor, the operation of the sensor may be compared and analyzed in real time using a reference sensor and/or a search gas. Through this, the change of the sensor may be detected, and a correction coefficient and/or a correction model for drift correction may be determined. Additionally, a method of adjusting a load resistance value may also be used to correct the output voltage value connected to the sensor.

However, as mentioned above, sensor drift correction may be affected not only by aging due to usage period, sensor lifespan, etc., but also by factors such as temperature and humidity due to changes in the measurement environment each time. Therefore, it may be problematic from a practical perspective to continuously perform the correction task repeatedly each time.

Therefore, such drift correction requires a method that may be continuously corrected by an automated protocol, and it may be essential to obtain stable features of a gas sensor-based system through this.

In other words, in the case of an e-nose system based on a gas sensor, improved detection capability and reliability may be secured by acquiring various features, i.e., characteristics, of the sensor while diversifying important feature characteristics. Through such feature diversification, the detection and pattern recognition capabilities of various gas components may be improved.

Additionally, drift compensation may be essential to detect and compensate for the drift phenomenon of the sensor to maintain the accuracy and reliability of the sensor. Such technical elements may help to improve the performance and practicality of gas sensor-based systems.

With reference to the above, the present disclosure proposes a method and a device/system that may accurately and reliably perform detection and analysis of various gas components by improving an e-nose system based on a gas sensor.

The proposed method in the present disclosure aims to solve the drift compensation problem of the aforementioned sensor and improve the performance of the system by accurately extracting characteristics for various gas components.

As described above, the existing gas sensor-based e-nose system has been utilized for detection and identification of specific gas components, but has a problem in that accuracy and reliability deteriorate over time due to the drift phenomenon of the sensor. To solve this problem, the present disclosure describes a method for maintaining consistent accuracy by compensating for sensor drift.

The proposed method in the present disclosure may provide robustness of drift correction and be applied to combinations of various sensors and features.

Specifically, with respect to the robustness of drift correction, the proposed method in the present disclosure relates to a method of maintaining the accuracy of a sensor by generating a compensated feature that is robust to sensor drift through a double cycling technique. Through this, stable sensor performance may be provided even when used for a long period of time.

Additionally, with respect to the combination of various sensors and features, the proposed method in the present disclosure relates to a method for improving the accurate pattern recognition capability for various gas components by extending the calibrated features acquired from each sensor by utilizing various combination methods. Through this, accurate identification of complex gas mixtures in real environments may be enabled.

That is, the proposed method in the present disclosure improves the performance and reliability of the system through drift correction and various sensor and feature combinations, and enables accurate and stable gas component analysis in various application fields.

Therefore, the present disclosure proposes a robust compensation method and various feature combinations for improving the performance and maintaining the accuracy of a gas sensor-based e-nose system, based on which accurate detection and identification capabilities for various gas components may be provided.

Below, a drift correction method based on double cycling and a method for combining various features are explained through specific examples.

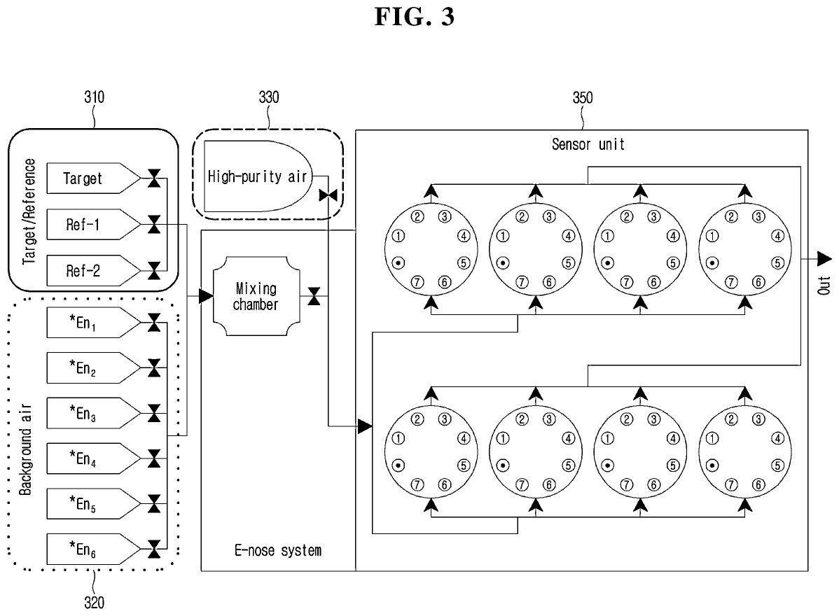

FIG. 3 illustrates a system configuration diagram for drift correction and feature combination according to an embodiment of the present disclosure.

Referring to FIG. 3, a system (e.g., a gas sensing hardware (H/W) system) required in connection with the proposed method in the present disclosure may be configured to include a first component (310) for injecting a target gas and/or a reference gas, a second component (320) for injecting a background gas, a third component (330) for injecting high-purity air, a fourth component (340) for mixing gases through a mixing chamber, and a fifth component (350) for performing sensing measurements on the mixed gas.

The first component (310) may correspond to a component for injecting a target gas and/or a reference gas that is a target of gas sensing.

The second component (320) may correspond to a component for injecting a background gas. For example, the second component (320) may be set to inject a background gas to dilute a target gas to be measured at a certain ratio or to reflect environmental factors or influences for a specific environment.

The third component (330) may correspond to a component for injecting high-purity air. For example, the third component (330) may be configured to measure the baseline sensor sensitivity (Rₐ) to obtain a sensor response (sensor response, Rₐ/R_{g}), and then inject high-purity air to clean the sensor again after obtaining the sensor response.

The fourth component (340) may be configured as a mixing chamber. For example, the target/reference gas, background gas, high purity air, etc., which are injected through the first component (310), the second component (320), and/or the third component (330), respectively, may be controlled through valves mounted on each line. The fourth component (340) may mix the injected gases at a certain ratio through the mixing chamber.

The fifth component (350) may perform measurements through a plurality of sensors (e.g., eight sensors mounted in each chamber in the case of FIG. 3) mounted in each of a plurality of chambers (e.g., eight in the case of FIG. 3). For example, each chamber may be configured as a uniform line, and measurements may be performed through even distribution through flow meter control for each line, etc.

The configuration (e.g., hardware configuration) described in FIG. 3 represents the configuration necessary to explain the proposed method in the present disclosure.

Hereinafter, key components and specific examples thereof are described in relation to the method proposed in the present disclosure.

First, a method for performing drift correction using the double cycling technique proposed in this disclosure is described.

In the proposed method of the present disclosure, drift-corrected data may be generated by collecting and utilizing measurement data of the first cycle and the second cycle for each sensor.

Specifically, in the first cycle, the initial signal of each sensor may be measured using a predefined reference gas.

In this process, the response of the reference gas may be used as a standard to accurately reflect the initial state and changed characteristics of the sensor. The reference gas may be applied differently depending on the sensor being used. For example, a gas reflecting the primary target provided in the manufacturer's datasheet for each sensor being used may be used as the reference gas.

Such initial data may provide important information indicating the sensitivity, response time, etc. of the sensor. Additionally, if a drift phenomenon occurs for the sensor, the drift phenomenon may also be reflected and measured in the initial state.

This step may be a critical step considering the long-term stability of the sensor, and the measurement accuracy of the first cycle may affect drift compensation and feature generation.

Next, in the second cycle, measurement data may be obtained using the actual target gas.

In the process, the response of the target gas may be measured to determine how the sensor responds to the response of the actual target gas. If a drift phenomenon occurs in the sensor, the drift phenomenon may be reflected in the actual target gas response.

Since the data from the second cycle represents the sensor response in a real environment, accurate data may be secured by including the effect even when drift occurs.

Through the measurement data of the first and second cycles as described above, the initial state and drift phenomenon of the sensor may be reflected, and data including all responses to the actual target gas may be secured. Through this, the purpose of the proposed method in the present disclosure may be achieved.

This process may improve the ability to provide improved drift correction and robust feature generation.

Additionally or alternatively, time variables for drift occurrence may be considered.

Specifically, since the measurement of the first cycle is always performed by measuring a reference gas set by the user (i.e., a reference gas that is already known), the measurement of the first cycle may be omitted if a certain amount of time has passed while measuring data by setting a time variable.

For example, if the time during which drift may occur is at least one week, the existing data stored as the measurement data of the first cycle may be utilized if the most recent measurement data is set to within one week.

Next, we describe a method for obtaining a drift-corrected robust feature proposed in the present disclosure.

A drift-corrected robust feature may be obtained by dividing the data acquired in the second cycle described above by the data acquired in the first cycle to calculate a ratio. Through this, the drift influence of the sensor is corrected, and an accurate feature may be generated.

For example, a robustly compensated feature corresponds to a feature obtained based on a ratio calculated by dividing the drift-compensated data by the initial data of the first cycle. The feature may accurately represent signal characteristics while compensating for sensor changes.

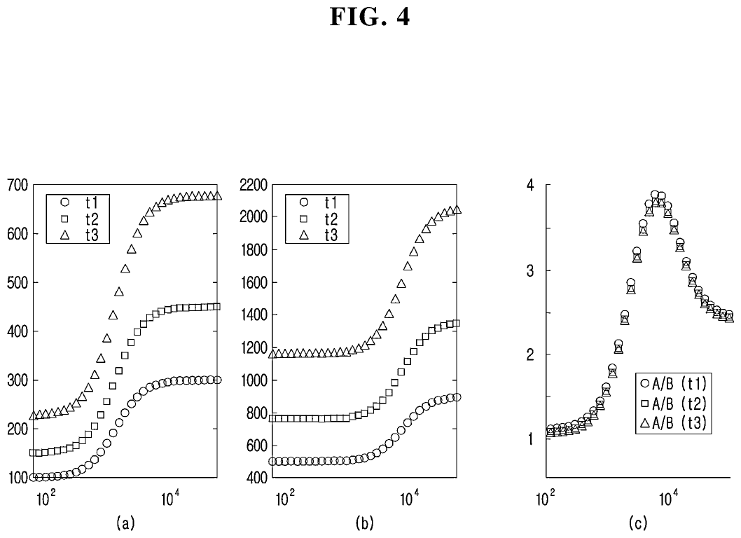

FIG. 4 illustrates simulation results showing the drift correction effect according to the double cycling technique according to an embodiment of the present disclosure.

Referring to FIG. 4, (a) of FIG. 4 shows the simulation results for the reference gas in the first cycle, (b) of FIG. 4 shows the simulation results for the target gas in the second cycle, and (c) of FIG. 4 shows the simulation results for the drift-corrected feature.

Each simulation result represents the state change phenomenon of sensor data at [t₁, t₂, t₃] defined by the state change due to each drift.

(c) of FIG. 4 shows the characteristics of features acquired by the double cycling technique for data in each state. Referring to this, it may be seen that the influence of drift that had a difference in [t₁, t₂, t₃] has been corrected, and that the feature pattern also has different characteristics from the original signal.

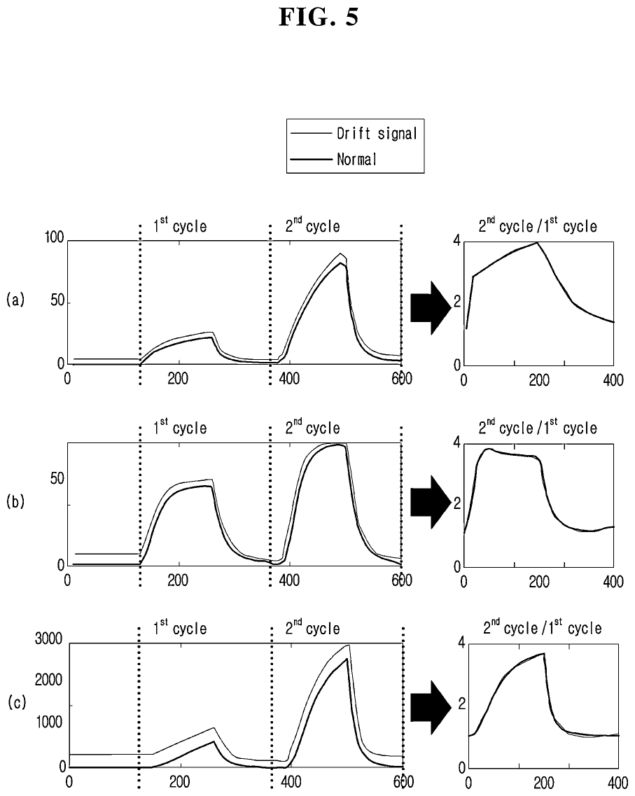

FIG. 5 illustrates the effect of drift correction based on double cycling obtained through data measured from a gas sensor according to an embodiment of the present disclosure.

Referring to FIG. 5, the left side of (a) of FIG. 5 shows a normal signal measured by sensor A and a signal due to drift, and the right side of (a) of FIG. 5 shows a robust feature characteristic corrected by the double cycling technique.

The left side of (b) of FIG. 5 shows the normal signal and the signal due to drift measured by sensor B, and the right side of (b) of FIG. 5 shows the robust feature characteristics corrected by the double cycling technique.

The left side of (c) of FIG. 5 shows the normal signal and the signal due to drift measured by sensor C, and the right side of (c) of FIG. 5 shows the robust feature characteristics corrected by the double cycling technique.

That is, FIG. 5 illustrates normal signals measured from three different sensors measured from a gas sensing system and signals when drift occurs.

In this regard, the signal by each sensor may be measured in the first cycle and the second cycle, and in the case of a state change due to each drift (i.e., a Drift signal), an upward offset may occur compared to a normal signal (i.e., Normal).

At this time, if the ratio calculated by dividing the data according to the target gas signal of the second cycle showing the size according to two different states by the data according to the reference gas of the first cycle is referred to, a corrected feature similar to the normal state may be obtained even when a drift occurs (e.g., see the right side of FIG. 5). In this case, since the pattern for the entire signal is corrected rather than simply obtaining a corrected value for one sample, there is a technical effect of being able to obtain a robust and unique feature.

Additionally, for each of the compensated characteristic features exhibiting unique characteristics acquired as described above, a method of generating another robust feature through a combination of the features may be considered.

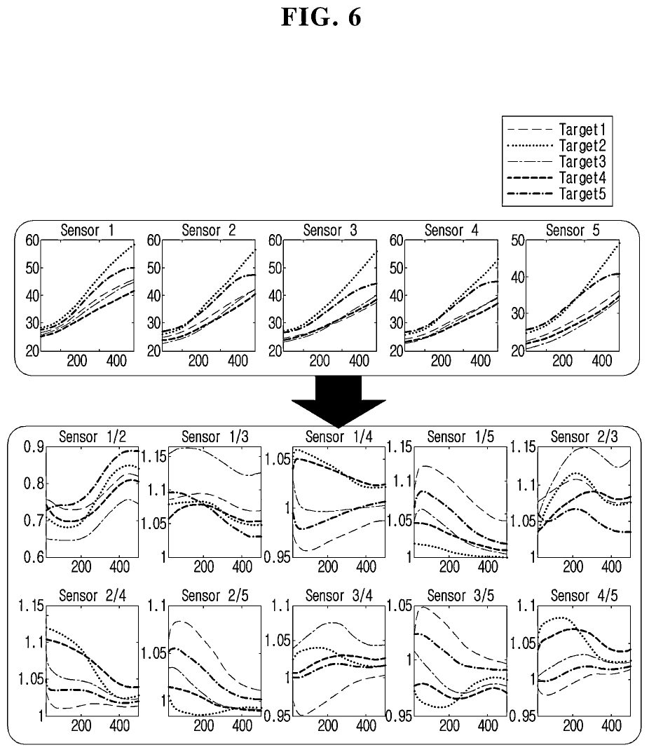

FIG. 6 illustrates feature diversification and expansion effects according to combinations of sensor characteristic features according to an embodiment of the present disclosure.

Referring to FIG. 6, when there are five sensors, one drift-corrected feature exhibiting a unique pattern may be obtained for each sensor through the double cycling technique described above in the present disclosure (i.e., a total of five drift-corrected features).

Various features may be generated through various combinations of the five features obtained in this way. For example, if two features are selected and combined from five features, ten features (i.e., ₅C₂) may be generated.

Below, a method for generating robust features by combining multiple features is explained through specific examples.

When generating robust features by selecting 2 out of n sensors, measurement combination data as many as ₙC₂ may be obtained. As a specific example, when expanding features by selecting 2 out of 5 sensors, 10 (i.e., ₅C₂) features may be generated.

Additionally, it may be extended to select not only 2 features but also 3, 4, 5, . . . , n features. In this case, the total number of features generated may be equal to Equation 1.

$$( \begin{matrix}N \\ 2\end{matrix} ) + ( \begin{matrix}N \\ 3\end{matrix} ) + … + ( \begin{matrix}N \\ N\end{matrix} ) \qquad [ Equation 1 ]$$

In Equation 1,

$$( \begin{matrix}N \\ r\end{matrix} )$$

represents the number of combinations of selecting r features out of N, and when generalizing this, the number of all possible combination features may be expressed as in Equation 2.

$${\sum }_{r = 2}^{N} ( \begin{matrix}N \\ r\end{matrix} ) \qquad [ Equation 2 ]$$

Additionally or alternatively, rather than simple operations such as multiplication or division as described above, various mathematical and statistical techniques may be used to combine multiple features into a robust feature, as in the following examples.

For example, new features may be generated based on exponential and logarithmic transformations. Specifically, new features may be generated by applying exponential or logarithmic functions to capture/generate nonlinear relationships between features.

For another example, new features may be generated based on polynomials. Specifically, the degree of an existing feature may be increased by generating a polynomial of the existing feature. This can model nonlinear relationships between features or capture/generate curved patterns.

For another example, a new feature may be generated based on the interaction between features. Specifically, an interaction feature may be generated by combining two or more features. For example, if there are features A and B, if a new feature is generated by a technique such as multiplying or dividing feature A and feature B, a specific pattern by the combination of feature A and feature B may be captured/generated.

As another example, new features may be generated based on dimensionality reduction. Specifically, by utilizing dimensionality reduction techniques such as principal component analysis (PCA) or linear discriminant analysis (LDA), features may be transformed into new axes and the dimensionality may be reduced.

As another example, new features may be created based on clustering. Specifically, clustering techniques may be used to cluster features and create new features that reflect the characteristics of data belonging to each cluster.

Another example is that new features may be generated using a neural network model. Specifically, a neural network model may be used to learn complex relationships between features, and new features may be generated through this. For example, a new feature may be obtained by compressing and then expanding features using an autoencoder.

As another example, new features may be generated based on a feature selection algorithm. Specifically, important features may be selected using a feature selection algorithm such as forward feature selection (FFS), recursive feature elimination (RFE), LASSO, etc., and new features may be generated using the selected features.

Additionally or alternatively, when the number of measurement samples for one cycle for a feature obtained as described above in the present disclosure is defined as S, the number of samples of the feature for each combination generated after performing the above-described process may be defined as in Equation 3.

$${S}_{ij}^{k} = {S}_{i}^{k} / {S}_{j}^{k} \qquad [ Equation 3 ]$$

In Equation 3, k represents the number of measurement samples, and i and j represent arbitrary numbers that may be selected from 1 to ₙC₂. At this time, with respect to ij, as many combinations as the number of cases corresponding to ₙC₂ may be generated.

Additionally or alternatively, a simplified system/hardware for the proposed method in the present disclosure may be configured.

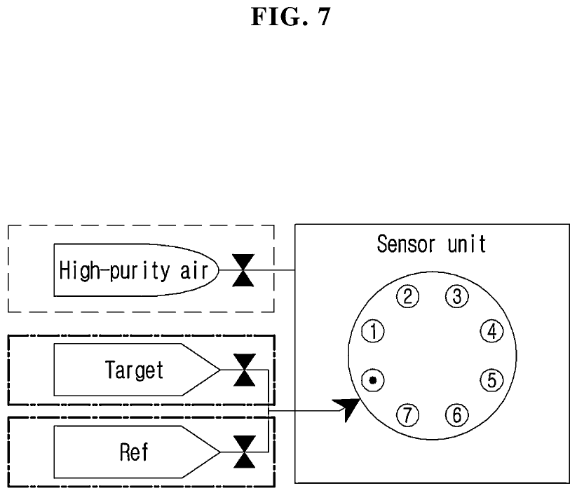

FIG. 7 illustrates a simplified system configuration implementing a double cycling technique and feature combination according to an embodiment of the present disclosure.

Referring to FIG. 7, for the proposed method in the present disclosure, when measuring a target gas to enable measurement using the double cycling technique, a gas sensor system capable of also measuring a reference gas in the first cycle needs to be implemented.

In this regard, a storage/space where the reference gas may be stored is required separately from the input inlet into which the target gas is injected, and a connection line or inlet for the reference gas may also be required.

Based on the configuration described above, the system configuration for the proposed method in the present disclosure may be manufactured in the form of a handheld device. Additionally, a system configuration in which the number of sensors, the number of types of target gases, the number of types of reference gases, etc. are expanded, as in the above-described FIG. 3, may also be possible.

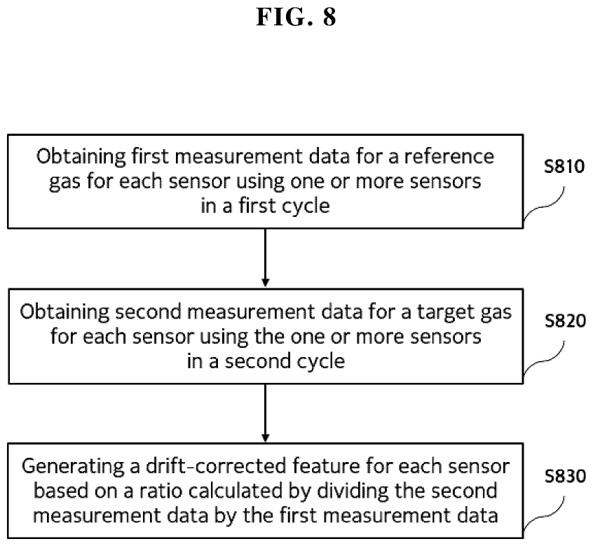

FIG. 8 illustrates an operational flowchart for a drift correction and feature combination method according to an embodiment of the present disclosure.

The operational flowchart described in FIG. 8 may be a specific example of a method for drift correction and generating robust features based on the double cycling technique described above in the present disclosure.

First, in the first cycle, first measurement data for a reference gas may be obtained for each sensor using one or more sensors (step S810).

Through this, measurement data related to the initial signal of the sensor may be obtained.

In this regard, each sensor may be configured/designed to be able to inject different types of reference gases.

Next, in the second cycle, second measurement data for the target gas may be obtained for each sensor using the one or more sensors (step S820).

In this regard, the second measurement data may include drift effects that may occur over time.

Thereafter, based on the ratio calculated by dividing the second measurement data by the first measurement data, a drift-corrected feature may be generated for each sensor (S830).

That is, by utilizing data measured in two cycles according to the double cycling technique, a more robust feature may be generated/obtained compared to data measured in one cycle.

In this regard, when multiple drift-corrected features for multiple sensors are generated, a method of generating new features based on these by considering various sensors and/or various characteristics may be additionally applied.

For example, when multiple drift-corrected features are generated for multiple sensors, one or more new features may be generated through combinations between the drift-corrected features.

As a concrete example, if there are n sensors and drift-corrected features for m sensors are selected for the combination, the maximum number of the one or more new features may correspond to ₙCₘ, where ₓC_{y} may represent a combination function for input x and input y.

Additionally or alternatively, when multiple drift-corrected features for multiple sensors are generated, an exponential function or a logarithmic function may be applied to the multiple drift-corrected features to generate one or more new features.

Additionally or alternatively, when multiple drift-corrected features for multiple sensors are generated, a polynomial for the multiple drift-corrected features may be generated. Then, based on the increased order according to the polynomial, one or more new features may be generated.

Additionally or alternatively, when multiple drift-corrected features for multiple sensors are generated, a predefined dimensionality reduction technique may be applied to the multiple drift-corrected features to generate one or more new features with reduced dimensionality.

Additionally or alternatively, when multiple drift-corrected features are generated for multiple sensors, the multiple drift-corrected features may be clustered by applying a predefined clustering technique. Then, one or more new features may be generated based on the characteristics of the data belonging to each cluster.

Additionally or alternatively, when multiple drift-corrected features for multiple sensors are generated, a predefined neural network model or a predefined feature selection algorithm may be applied to the multiple drift-corrected features to generate one or more new features.

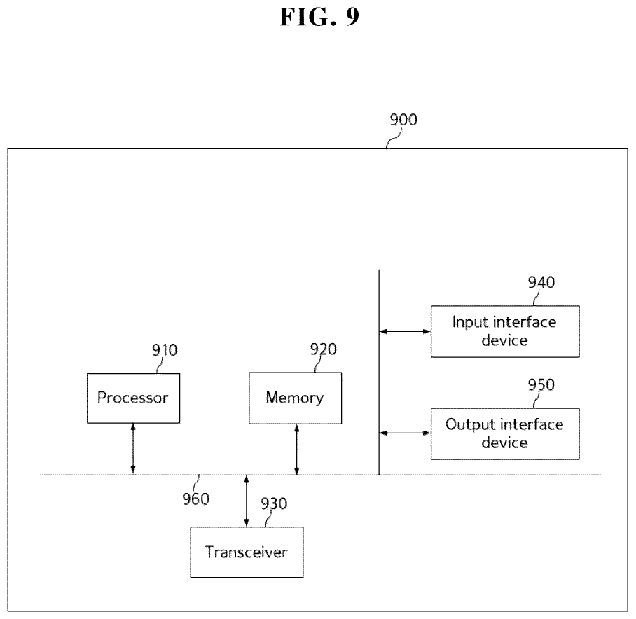

FIG. 9 is a block diagram illustrating an apparatus according to an embodiment of the present disclosure.

Referring to FIG. 9, a device (900) may represent a device implementing a double cycling technique for drift correction and a method for performing feature-to-feature combination as described in the present disclosure.

For example, the device (900) may generally support/perform functions such as performing sensor data correction through a double cycling technique, and generating new data/features through a combination of data/features.

The device 900 may include at least one of a processor 910, a memory 920, a transceiver 930, an input interface device 940, and an output interface device 950. Each of the components may be connected by a common bus 960 to communicate with each other. In addition, each of the components may be connected through a separate interface or a separate bus centering on the processor 910 instead of the common bus 960.

The processor 910 may be implemented in various types such as an application processor (AP), a central processing unit (CPU), a graphic processing unit (GPU), etc., and may be any semiconductor device that executes a command stored in the memory 920. The processor 910 may execute a program command stored in the memory 920. The processor (910) may be configured to implement a method and device for performing a double cycling technique and feature combination for drift correction based on the above-described FIGS. 1 to 8.

And/or, the processor 910 may store a program command for implementing at least one function for the corresponding modules in the memory 920 and may control the operation described based on FIGS. 1 to 8 to be performed.

The memory 920 may include various types of volatile or non-volatile storage media. For example, the memory 920 may include read-only memory (ROM) and random access memory (RAM). In an embodiment of the present disclosure, the memory 920 may be located inside or outside the processor 910, and the memory 920 may be connected to the processor 910 through various known means.

The transceiver 930 may perform a function of transmitting and receiving data processed/to be processed by the processor 910 with an external device and/or an external system.

The input interface device 940 is configured to provide data to the processor 910.

The output interface device 950 is configured to output data from the processor 910.

According to the present disclosure, a method and device for performing sensor drift correction based on double cycling measurement may be provided.

According to the present disclosure, a method and device may be provided that may improve the performance and reliability of a system by performing sensor drift correction based on double cycling measurement and generating various characteristic features based thereon.

According to the present disclosure, a method and device are provided that may solve a drift compensation problem of a sensor and accurately extract characteristics for various gas components to improve the performance and reliability of a system.

Specifically, the proposed method in the present disclosure is a method for obtaining accurate detection and pattern recognition capabilities of various gas components in a gas sensor-based e-nose system.

The proposed method in the present disclosure may improve the performance and reliability of the system through the process of compensating for drift phenomenon that may occur in existing gas sensor technologies and generating robust features.

In this regard, the proposed method in the present disclosure includes a double cycling technique that utilizes the initial data acquired in the first cycle to correct the data acquired in the second cycle. Through this, even if a drift phenomenon occurs, it may be included in the initial data and corrected, and thus robust data may be generated.

Additionally, the proposed method in the present disclosure includes a method of generating extended features of different patterns through various combinations (e.g., mathematical techniques, statistical techniques, etc.) between acquired features, and high-dimensional data analysis based on this may be possible. Through this, various unique features may be acquired, and based on this, the response characteristics of the sensor may be accurately detected. In addition, the recognition ability for various gas mixtures may be improved, and the detection performance of the system may be improved.

That is, according to the embodiment of the present disclosure, comprehensive data may be acquired considering the initial state and drift phenomenon of the sensor, so that reliable results may be provided compared to existing technologies, and based on this, there is a technical effect of providing improved detection capability and reliability.

The components described in the example embodiments may be implemented by hardware components including, for example, at least one digital signal processor (DSP), a processor, a controller, an application-specific integrated circuit (ASIC), a programmable logic element, such as an FPGA, GPU other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers.

A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors. Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment.

Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Accordingly, it is intended that this disclosure embrace all other substitutions, modifications and variations belong within the scope of the following claims.

What is claimed is:

1. A method for performing drift correction according to gas sensor measurement, the method comprising:
obtaining first measurement data for a reference gas for each sensor using one or more sensors in a first cycle;
obtaining second measurement data for a target gas for each sensor using the one or more sensors in a second cycle; and
generating a drift-corrected feature for each sensor based on a ratio calculated by dividing the second measurement data by the first measurement data.

2. The method of claim 1, further comprising:
generating one or more new features through combinations between drift-corrected features, in a case that a plurality of drift-corrected features for a plurality of sensors are generated.

3. The method of claim 2,
wherein, when n sensors exist and drift-corrected features for m sensors are selected for the combination, a maximum number of the one or more new features corresponds to ₙCₘ, and
wherein ₓC_{y} represents a combination function for input x and input y.

4. The method of claim 1, further comprising:
in a case that a plurality of drift-corrected features are generated for a plurality of sensors, generating one or more new features by applying an exponential function or a logarithmic function to the plurality of drift-corrected features.

5. The method of claim 1, further comprising:
in a case that a plurality of drift-corrected features are generated for a plurality of sensors, generating a polynomial for the plurality of drift-corrected features; and
generating one or more new features based on an increased order according to the polynomial.

6. The method of claim 1, further comprising:
in a case that a plurality of drift-corrected features are generated for a plurality of sensors, generating one or more new features with reduced dimension by applying a pre-defined dimension reduction scheme to the plurality of drift-corrected features.

7. The method of claim 1, further comprising:
in a case that a plurality of drift-corrected features are generated for a plurality of sensors, clustering the plurality of drift-corrected features by applying a pre-defined clustering scheme; and
generating one or more new features based on characteristics of data belonging to each cluster.

8. The method of claim 1, further comprising:
in a case that a plurality of drift-corrected features are generated for a plurality of sensors, generating one or more new features by applying a pre-defined neural network model or a pre-defined feature selection algorithm to the plurality of drift-corrected features.

9. The method of claim 1,
wherein each sensor is configured to be able to inject different types of reference gases.

10. An apparatus of performing drift correction according to gas sensor measurement, the apparatus comprising:
at least one processor and at least one memory,
wherein the processor is configured to:
obtain first measurement data for a reference gas for each sensor using one or more sensors in a first cycle;
obtain second measurement data for a target gas for each sensor using the one or more sensors in a second cycle; and
generate a drift-corrected feature for each sensor based on a ratio calculated by dividing the second measurement data by the first measurement data.

11. The apparatus of claim 10,
wherein the processor is configured to:
generate one or more new features through combinations between drift-corrected features, in a case that a plurality of drift-corrected features for a plurality of sensors are generated.

12. The apparatus of claim 11,
wherein, when n sensors exist and drift-corrected features for m sensors are selected for the combination, a maximum number of the one or more new features corresponds to ₙCₘ, and
wherein ₓC_{y} represents a combination function for input x and input y.

13. The apparatus of claim 10,
wherein the processor is configured to:
in a case that a plurality of drift-corrected features are generated for a plurality of sensors, generate one or more new features by applying an exponential function or a logarithmic function to the plurality of drift-corrected features.

14. The apparatus of claim 10,
wherein the processor is configured to:
in a case that a plurality of drift-corrected features are generated for a plurality of sensors, generate a polynomial for the plurality of drift-corrected features; and
generate one or more new features based on an increased order according to the polynomial.

15. The apparatus of claim 10,
wherein the processor is configured to:
in a case that a plurality of drift-corrected features are generated for a plurality of sensors, generate one or more new features with reduced dimension by applying a pre-defined dimension reduction scheme to the plurality of drift-corrected features.

16. The apparatus of claim 10,
wherein the processor is configured to:
in a case that a plurality of drift-corrected features are generated for a plurality of sensors, cluster the plurality of drift-corrected features by applying a pre-defined clustering scheme; and
generate one or more new features based on characteristics of data belonging to each cluster.

17. The apparatus of claim 10,
wherein the processor is configured to:
in a case that a plurality of drift-corrected features are generated for a plurality of sensors, generate one or more new features by applying a pre-defined neural network model or a pre-defined feature selection algorithm to the plurality of drift-corrected features.

18. The apparatus of claim 10,
wherein each sensor is configured to be able to inject different types of reference gases.

19. One or more non-transitory computer readable medium storing one or more instructions,
wherein the one or more instructions are executed by one or more processors and control an apparatus for performing drift correction based on gas sensor measurements to:
obtain first measurement data for a reference gas for each sensor using one or more sensors in a first cycle;
obtain second measurement data for a target gas for each sensor using the one or more sensors in a second cycle; and
generate a drift-corrected feature for each sensor based on a ratio calculated by dividing the second measurement data by the first measurement data.

20. The computer readable medium of claim 19,
wherein the one or more instructions are executed by one or more processors to control a device for performing drift correction according to gas sensor measurements:
in a case that a plurality of drift-corrected features for a plurality of sensors are generated, generate one or more new features through combinations of drift-corrected features.
